(19) United States
(12) Patent Application Publication
(10) Pub. No.: US 2016/0175564 A1
(43) Pub. Date: Jun. 23, 2016

(54) GUIDEWIRE TORQUE HANDLE

(71) Applicant: VASCULAR IMAGING CORPORATION, Rancho Cordova, CA (US)

(72) Inventor: Michael J. Eberle, Fair Oaks, CA (US)

(21) Appl. No.: 14/909,937

(22) PCT Filed: Aug. 5, 2014
(86) PCT No.: PCT/US2014/049713

§ 371 Date: Feb. 3, 2016

Related U.S. Application Data
(60) Provisional application No. 61/862,184, filed on Aug. 5, 2013.

(51) Int. Cl. A61M 25/09

(52) U.S. Cl. CPC A61M 25/09041; A61M 2025/09116

(57) ABSTRACT

This document describes techniques for gripping a guidewire (12). In one example, a device (30) includes an elongated handle (14) defining a handle lumen extending from a distal handle end to a proximal handle end, a handle insert (32,34,36) having a length and being configured to be inserted into the handle lumen, wherein a handle insert material is more compliant than a handle material, and wherein the handle insert defines a guidewire lumen configured to receive a guidewire, and a cap (16) configured to compress the handle insert to grip the guidewire along a substantial portion of the length of the handle insert.

This application claims the benefit of priority to U.S. Provisional Application No. 61/862,184, filed on Aug. 5, 2013, the entire content of which is incorporated herein by reference in its entirety.

### TECHNICAL FIELD

This document pertains generally to medical devices and, in particular, to devices for handling guidewires.

### BACKGROUND

A guidewire is an elongated wire or fiber having a small diameter, e.g., about 0.014 inches, which may be inserted into a body lumen of a patient.

Guidewire torque handles are used to provide large diameter surfaces for the application of torque to the small diameter guidewires, which may be essential to enable an operator, physician, or clinician, to accurately turn the guidewire to steer it to a desired location within the body. Guidewire torque handles may be especially helpful when using surgical gloves.

In some implementations, the torque handle may be temporarily affixed to the guidewire by mechanical means. Various torque handle designs are available. Most commonly, the torque handle is made from low cost injection molded plastic components, combined with metallic pin vice jaws. When the nose cone of the torque handle is tightened onto the guidewire, the metallic pin vice jaws engage the surface of the guidewire and grip in four longitudinal locations parallel with the guidewire axis. The four points of the pin vice jaws typically sink into the surface of the guidewire, which is normally made from a solid core at the attachment location, with or without coatings. While the pin vice jaws may cause some damage to the guidewire surface, it is usually not problematic and does not interfere with normal guidewire use.

### OVERVIEW

While standard guidewires are made with solid cores in the proximal shaft areas, guidewires with advanced sensing capabilities incorporate signal conductors by using a tubular proximal shaft. The signal conductors may be electrical or optical, and are typically threaded coaxially within the hollow tube. If the tube has a thin wall, the standard torque handle pin vice jaws may damage the wall, may damage the signal conductors, and may render the sensing device inoperable. Other sensor guidewires may incorporate signal conductors along the outside surface of a solid core proximal shaft. In this case, the pin vice jaws of the torque handle may engage the signal conductors and cause lasting damage, thereby rendering the sensing guidewire inoperable. It is an object of the present invention to provide a low cost guidewire torque handle that can be temporarily attached to a guidewire without causing damage to any signal conductors of the guidewire.

In one example, the disclosure is directed to a device for gripping a guidewire. The device comprises an elongated handle defining a handle lumen extending from a distal handle end to a proximal handle end, a handle insert having a length and being configured to be inserted into the handle lumen, wherein a handle insert material is more compliant than a handle material, and wherein the handle insert defines a guidewire lumen configured to receive a guidewire, and a cap configured to compress the handle insert to grip the guidewire along a substantial portion of the length of the handle insert.

In another example, the disclosure is directed to a device for gripping a guidewire. The device comprises an elongated handle defining a handle lumen extending from a distal handle end to a proximal handle end, the handle lumen having a distal portion and a proximal portion, the distal portion having a first lateral dimension and the proximal portion having a second lateral dimension different from the first lateral dimension. The device further comprises a first handle insert configured to be inserted into the handle lumen, wherein a portion of the first handle insert is configured to extend from the distal portion of the handle to the proximal portion of the handle. The device further comprises a second handle insert configured to be inserted into the handle lumen, wherein the second handle insert is cylindrical and is disposed distally adjacent to the first handle insert. The device further comprises a third handle insert configured to be inserted partially into the handle lumen, wherein the third handle insert is disposed distally adjacent to the second handle insert, and a cap configured to be disposed about a portion of an outer surface of the distal handle end and about a distal portion of the third handle insert, wherein the first, second, and third handle inserts each define a guidewire lumen, the guidewire lumen configured to receive a guidewire, wherein a distal end of the cap is configured to receive the guidewire and direct the guidewire into the guidewire lumen, wherein the second handle insert includes a material that is more compliant than a first handle insert material or a third handle insert material, and

wherein the second handle insert is configured to grip the guidewire.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective, exploded view of a PRIOR ART device for gripping a guidewire.

FIG. 2 is a perspective, exploded view of a device for gripping a guidewire, in accordance with this disclosure.

FIG. 3 is a cross-sectional view of the elongated handle of FIG. 2.

FIG. 4 is a cross-sectional view of the device of FIG. 2.

FIG. 5 is a cross-sectional view of another example configuration of a device for gripping a guidewire, in accordance with this disclosure.

FIG. 6 is a cross-sectional view of another example configuration of a device for gripping a guidewire, in accordance with this disclosure.

### DETAILED DESCRIPTION

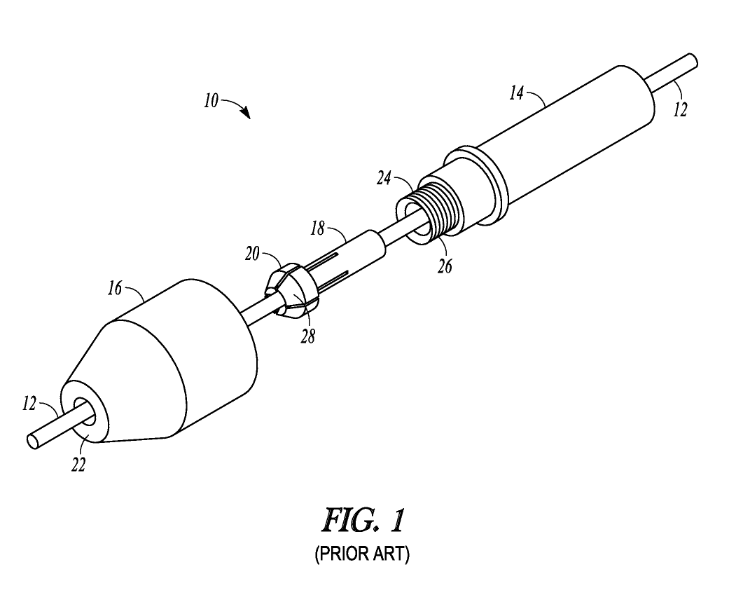

FIG. 1 is a perspective, exploded view of a PRIOR ART device 10 for gripping a guidewire 12. The device 10 is available from Merit Medical Systems, Inc. and includes an elongated handle 14, a cap 16, and a metallic insert 18 that includes pin vice jaws 20 configured to engage a surface of the guidewire 12. The metallic insert 18 is configured to be inserted into the tubular handle 14.

The guidewire 12 is inserted through a proximal end 22 of the cap 16, through a guidewire lumen defined by and extending through the metallic insert 18, and through a handle lumen defined by the handle 14. The cap 16 includes internal screw threads (not shown) and is configured to receive a distal end 24 of the tubular handle 14, which includes external screw threads 26. The internal and external screw threads form a threaded engagement between the handle 14 and the cap 16.

As mentioned above, the metallic insert 18 typically includes pin vice jaws 20, e.g., having four points 28, that can sink into the surface of a guidewire. Some guidewires may include advanced sensing capabilities that incorporate signal conductors by using a tubular proximal shaft. The signal conductors may be electrical or optical, and are typically threaded coaxially within the hollow tube. If the tube has a thin wall, the standard torque handle pin vice jaws may damage the wall, may damage the signal conductors, and may render the sensing device inoperable.

Other sensor guidewires may incorporate signal conductors along the outside surface of a solid core proximal shaft. In this case, the pin vice jaws of the torque handle may engage the signal conductors and cause lasting damage, thereby rendering the sensing guidewire inoperable. It is an object of the present invention to provide a low cost guidewire torque handle that can be temporarily attached to a guidewire without causing damage to any signal conductors of the guidewire.

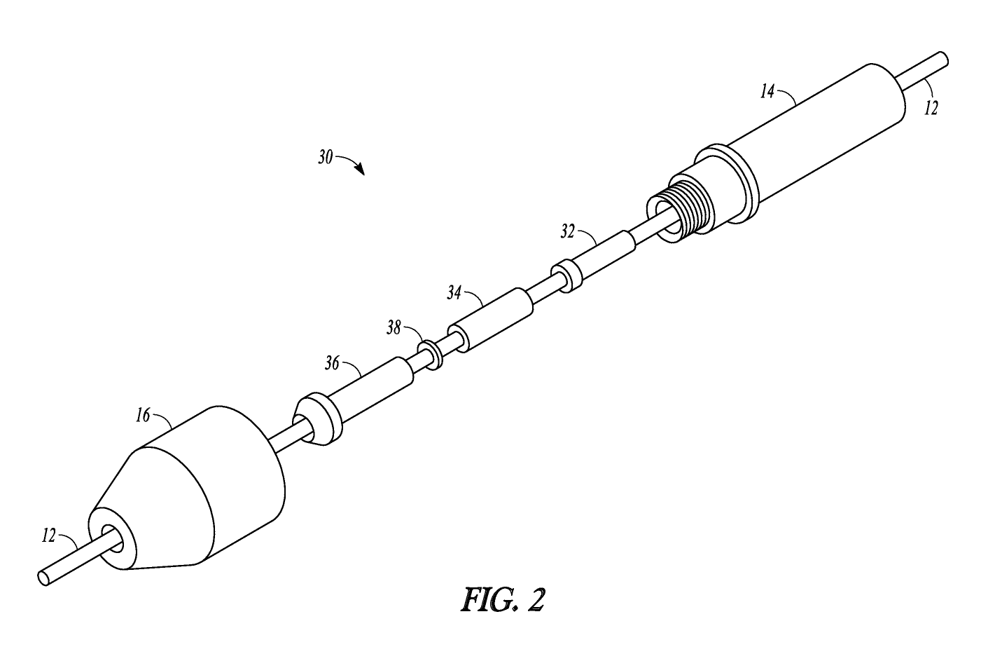

FIG. 2 is a perspective, exploded view of a device 30 for gripping a guidewire 12, in accordance with this disclosure. As described in more detail below, the device 30, e.g., a guidewire torque handle, may be designed to use a low impact, compliant material to grip the surface of the guidewire 12 extending through the device 30. In this manner, the device 30 can be temporarily attached to the guidewire 12 without causing damage to any signal conductors of the guidewire 12. One non-limiting example of a guidewire 12, e.g., a guidewire that includes advanced sensing capabilities, that can be used in conjunction with the devices of this disclosure is described in detail in U.S. Pat. No. 7,245,789 to Bates et al., entitled “Systems and Methods for Minimally-Invasive Optical Acoustic Imaging,” the entire contents incorporated by reference herein.

In FIG. 2, the device 30 may include the elongated handle 14 and the cap 16 of the prior art device 10 of FIG. 1. In contrast to the prior art device 10 of FIG. 1, the device 30 of FIG. 2, does not include the metallic insert 18. Rather, in some examples, the metallic insert 18 of FIG. 1 may be replaced by three inserts, namely first (or proximal) handle insert 32, second handle insert 34, and third (or distal) handle insert 36. Each of the first, second, and third handle inserts define a portion of a guidewire lumen (depicted at 56 in FIG. 4).

In some examples, the device 30 may include a washer 38 positioned between the second handle insert 34 and the third handle insert 36. Turning of the cap 16 may cause the third handle insert 36 to turn. To prevent or mitigate a transfer of rotational energy to the second handle insert 34, the washer 38 may be included.

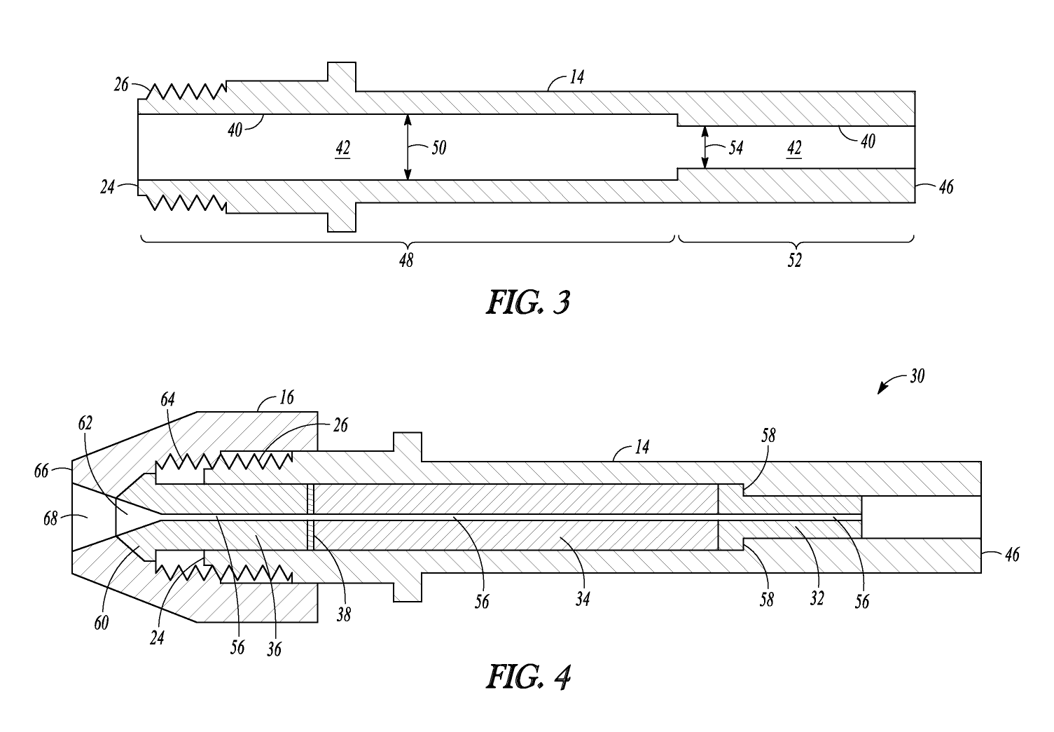

In accordance with this disclosure and as described in more detail below, one of the handle inserts, e.g., the second handle insert 34 of FIG. 2, may include a compliant material that may compress and grip the guidewire 12 when the cap 16 is proximally advanced, e.g., via rotation. The material property of the second handle insert 34 may be more compliant than adjacent insert material property, e.g., first handle insert 32, or adjacent handle material property, e.g., an interior surface 40 of handle 14 (FIG. 3). In one example implementation, the second handle insert 34 is cylindrically shaped and substantially uniform, e.g., without slotted or other features that define a chuck or jaws (as in FIG. 1).

The proximal advancement of the cap causes a compressive force to be imparted onto the second handle insert 34, e.g., a rubber tube, which causes the guidewire lumen to collapse around and securely grip the guidewire 12, distributing the gripping force along a significantly larger surface area than the vice grips. To release the guidewire 12, the cap 16 may be distally advanced, e.g., via rotation, which causes the compliant material of the second handle insert 34 to release its grip. The material of the second handle insert 34 has elastic properties and, as such, is not permanently deformed. Thus, the second handle insert 34 may be compressed again for multiple uses.

FIG. 3 is a cross-sectional view of the elongated handle 14 of FIG. 2. As seen in FIG. 3, an interior surface 40 of the elongated handle 14 defines a handle lumen 42 that extends longitudinally from a distal handle end 24 to a proximal handle end 46. The handle lumen 42 has a distal portion 48 with a first lateral internal dimension 50, e.g., diameter, and a proximal portion 52 with a second lateral internal dimension 54, e.g., diameter, different from the first lateral dimension 50.

FIG. 4 is a cross-sectional view of the device 30 of FIG. 2. In the example configuration depicted in FIG. 4, the device 30 includes three inserts, namely first handle insert 32, second handle insert 34, and third handle insert 36. Each of the first, second, and third handle inserts define a guidewire lumen 56.

The first handle insert 32 is configured to be inserted into the handle lumen 42, where a portion of the first handle insert 32 is configured to extend from the distal portion 48 to the proximal portion 52. As seen in FIG. 2, the first handle insert 32 may abut a portion 58 of the interior surface 40 of the handle lumen 42 where the first lateral dimension (shown at 50 in FIG. 3) reduces to the second lateral dimension (shown at 54 in FIG. 3). Such abutment may prevent the first handle insert 32 from moving proximally toward proximal handle end 46.

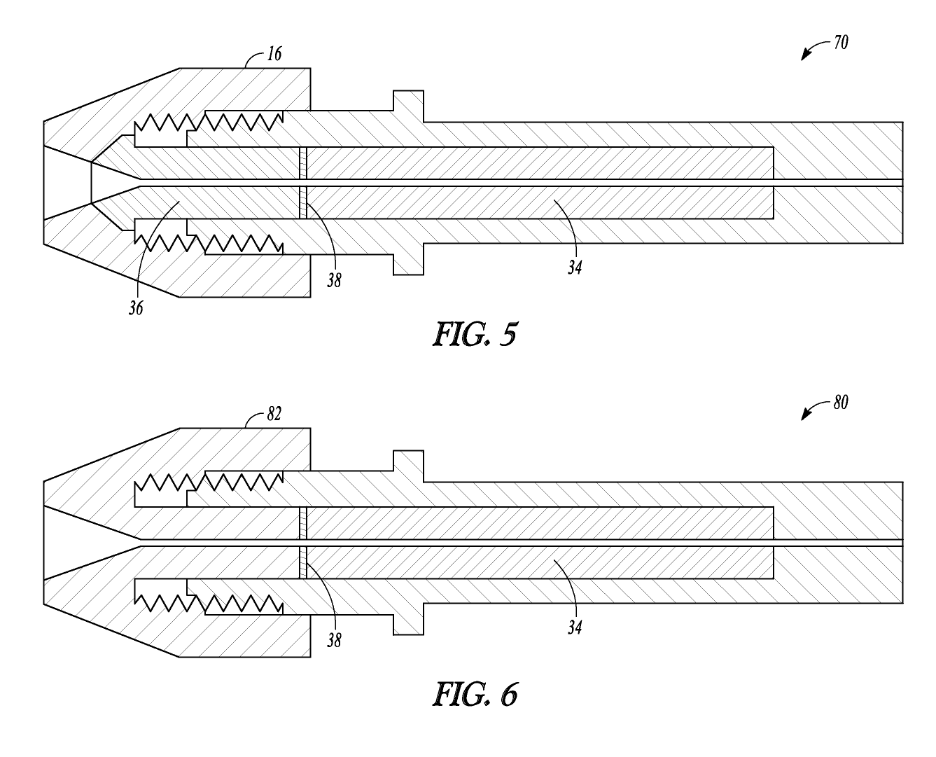

In another example configuration, the first handle insert 32 may form a part of the handle 14 rather than being a separate piece that is inserted into the handle lumen 42. Such a configuration is described in detail below with respect to FIG. 5.

Referring again to FIG. 4, the device 30 may further include a second handle insert 34. The second handle insert 34 may be made of a compliant material and may be generally cylindrical and configured to be inserted into the handle lumen 42. The second handle insert 34 may be disposed proximally adjacent to the first handle insert 32. In one example, the second handle insert 34 may be a rubber tube with relatively high precision. In another example, the second handle insert 34 may be a silicone tube.

The device 30 of FIG. 4 may further include a third handle insert 36. The third handle insert 36 may be configured to be inserted partially into the handle lumen 42 and disposed distally adjacent to the second handle insert 34. In some examples, the device 30 may include a washer 38 positioned between the second handle insert 34 and the third handle insert 36. As seen in FIG. 4, a distal end 60 of the third handle insert 36 may extend distally from the distal end 24 of the handle 14. In some example configurations, the distal end 60 of the third handle insert 36 can define a conical region 62 to aid in advancing the guidewire 12 into the guidewire lumen 56.

In one example configuration, the second handle insert 34 may include a material that is more compliant than a material of the first handle insert 32, e.g., plastic, a material of the third handle insert 36, e.g., plastic, or a material of the handle 14. The second handle insert 34 may be extruded or molded, for example.

As indicated above, the device 30 may further include the cap 16 (e.g., nose cone). The cap 16 may be configured to be disposed about a portion of an outer surface of the distal handle end 24 and about the distal end 60 of the third handle insert 36. Similar to the prior art device 10 described above in FIG. 1, in one example, the cap 16 may include internal screw threads 64 and may be configured to receive the distal end 24 of the handle 14, which includes external screw threads 26. The internal and external screw threads form a threaded engagement between the cap 16 and the handle 14.

In some examples, the cap 16 may include external screw threads (not depicted) and the distal handle end 24 may have internal screw threads (not depicted). In such a configuration, the distal handle end 24 may be configured to receive the external screw threads of the cap 16.

When the handle 14 is located at the desired position along the length of the guidewire 12, the guidewire 12 may be gripped by turning the cap 16 onto the handle 14. As the cap 16 is advanced onto the handle 14, the third handle insert 36 is longitudinally advanced proximally, which exerts a force against the compressible second handle insert 34. The first handle insert 32 prevents the longitudinal advancement of the second handle insert 34, which causes the second handle insert 34 to compress and, since the diameter of the bore 50 is a relatively close tolerance fit to the outer diameter of insert 34, the force causes the guidewire lumen 56 to collapse around the guidewire 12.

Turning the cap 16 may cause a compressive force to be imparted onto the second handle insert 34, which, in turn, causes the guidewire lumen 56 to collapse around the guidewire 12. When the guidewire lumen 56 is fully collapsed, an inner surface of the second handle insert 34 that defines a portion of the guidewire lumen 56 makes a friction grip on an outer surface of the guidewire 12 along a substantial portion of a length of the handle insert 34. The friction grip is distributed relatively evenly over a large surface area, which minimizes the gripping force on any particular component such as the coating, the core wire or hollow tubes, and therefore there is no noticeable damage to the guidewire 12 or any of the signal conductors located internally or on the surface of the guidewire 12. It may be desirable to include a stop mechanism to prevent over tightening of the cap 16 when the guidewire lumen 56 of the second handle insert 34 is completely collapsed.

In some examples, it may be desirable to minimize the number of turns needed to accomplish a complete collapse of the guidewire lumen 56 along a substantial portion of the length of the second handle insert 34, e.g., the entire length of the second handle insert 34. As such, it may be desirable for the guidewire lumen 56 of the second handle insert 34 to have an internal lateral dimension, e.g., diameter, that clears an outer diameter of the guidewire 12 as closely as reasonably possible. In addition, the volume of the second handle insert 34 may be sufficiently large that a small compression along its longitudinal axis (which is parallel to a longitudinal axis of the handle 14) may collapse the guidewire lumen 56 around the guidewire 12, thereby securely gripping the guidewire 12 along the length of the second handle insert 34. In this manner, the guidewire 12 may be gripped by the compliant second handle insert 34 without damage to any guidewire components and signal conductors.

A distal end 66 of the cap 16 is configured to receive the guidewire 12 and direct the guidewire 12 into the guidewire lumen 56. In one example configuration, the distal end 66 of the cap 16 may define a frustoconical region 68. Together, the frustoconical region 68 of the cap 16 and the conical region 62 of the third handle insert 36 may form a conical region to direct the guidewire 12 into the guidewire lumen 56.

In one example configuration, the cap 16 is configured to receive the proximal end 24 of the handle 14, which includes external screw threads 26.

FIG. 5 is a cross-sectional view of another example configuration of a device 70 for gripping a guidewire 12, in accordance with this disclosure. In FIG. 5, the first handle insert 32 of FIG. 4 forms a part of the handle 14 rather than being a separate piece that is inserted into the handle lumen 42 of FIG. 3. As such, the configuration shown in FIG. 5 includes two inserts, namely second handle insert 34 and third handle insert 36. Such a configuration may advantageously reduce the number of components to be manufactured.

FIG. 6 is a cross-sectional view of another example configuration of a device 80 for gripping a guidewire 12, in accordance with this disclosure. In FIG. 6, the cap 16 of FIG. 5 may be integrally formed with the third handle insert 36 of FIG. 4, thereby forming a single piece, shown as a cap 82. Such a configuration may advantageously further reduce the number of components to be manufactured. In some example configurations (not depicted), the device may include the integrated cap 80 of FIG. 6, and both the first and second handle inserts 32, 34 of FIG. 4.

Each of these non-limiting examples described above may stand on its own, or may be combined in various permutations or combinations with one or more of the other examples.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments may be combined with each other in various combinations or permutations. The scope of the invention should be determined with reference to the appended claims, along with the full scope of equivalents to which such claims are entitled.

1. A device for gripping a guidewire, the device comprising:
an elongated handle defining a handle lumen extending from a distal handle end to a proximal handle end;
a cylindrical handle insert having a length and being configured to be inserted into the handle lumen, wherein a handle insert material is more compliant than a handle material, and wherein the handle insert defines a guidewire lumen configured to receive a guidewire; and
a cap configured to be advanced onto the handle and to compress the handle insert to grip the guidewire along the entire length of the handle insert.

2. The device of claim 1, comprising:
a distal handle insert configured to be inserted at least partially into the handle lumen, wherein the distal handle insert is disposed distally adjacent to the handle insert.

3. The device of claim 2, comprising:
a washer configured to he disposed between the handle insert and the distal handle insert.

4. The device of claim 1, comprising:
a proximal handle insert configured to be inserted into the handle lumen, wherein the proximal handle insert is disposed proximally adjacent to the handle insert.

5. The device of claim 4, wherein the proximal portion of the handle has a first lateral dimension and the distal portion of the handle has a second lateral dimension different from the first lateral dimension, and wherein a portion of the proximal handle insert is configured to extend from the distal portion of the handle to the proximal portion of the handle.

6. The device of claim 1, wherein a portion of the cap is configured to extend proximally into the handle lumen.

7. The device of claim 1, wherein the handle insert is cylindrical.

8. The device of claim 1, wherein the cap is configured to be disposed about a portion of an outer surface of the distal portion of the handle.

9. A device for gripping a guidewire, the device comprising:
an elongated handle defining a handle lumen extending from a distal handle end to a proximal handle end, the handle lumen having a distal portion and a proximal portion, the distal portion having a first lateral dimension and the proximal portion having a second lateral dimension different from the first lateral dimension;
a first handle insert configured to be inserted into the handle lumen, wherein a portion of the first handle insert is configured to extend from the distal portion of the handle to the proximal portion of the handle;
a second handle insert configured to be inserted into the handle lumen, wherein the second handle insert is cylindrical and is disposed distally adjacent to the first handle insert;
a third handle insert configured to be inserted partially into the handle lumen, wherein the third handle insert is disposed distally adjacent to the second handle insert; and
a cap configured to be disposed about a portion of an outer surface of the distal handle end and about a distal portion of the third handle insert,
wherein the first, second, and third handle inserts each define a guidewire lumen, the guidewire lumen configured to receive a guidewire,
wherein a distal end of the cap is configured to receive the guidewire and direct the guidewire into the guidewire lumen,
wherein the second handle insert includes a material that is more compliant than a first handle insert material or a third handle insert material, and
wherein the second handle insert is configured to grip the guidewire.

10. The device of claim 9, comprising:
a washer configured to be disposed between the first handle insert and the second handle insert.

11. The device of claim 9, wherein the second handle insert is configured to compress and grip the guidewire along the entire length of the second handle insert.
